(19) United States
(12) Patent Application Publication
(10) Pub. No.: US 2022/0338873 A1
(43) Pub. Date: Oct. 27, 2022

(54) METHOD AND APPARATUS FOR HOLDING SURGICAL SUTURES

(71) Applicant: Jessica Adams, Sandy, UT (US)

(72) Inventor: Jessica Adams, Sandy, UT (US)

(21) Appl. No.: 17/334,047

(22) Filed: May 28, 2021

Related U.S. Application Data
(60) Provisional application No. 63/178,654, filed on Apr. 23, 2021.

(51) Int. Cl. A61B 17/06; A61B 90/94

(52) U.S. Cl. CPC A61B 17/06114; A61B 90/94; A61B 2017/06147

(57) ABSTRACT

A method and apparatus for holding a suture needle may include a suture needle pad disposed on a suture tray for holding the needle and suture originally contained with the suture tray. The suture tray/suture tray cover may include identifying information for the needle disposed in the suture needle pad and the associate suture thread.

### BACKGROUND

### State of the Art

The present invention relates to a method and apparatus for holding a surgical suture.

### Field of Art

Medical procedures have advanced to the point where doctors routinely conduct complex surgeries on patients for a variety of reasons. In some cases the surgeries involve removal of cancerous or otherwise diseased tissues. In other cases, the surgery is meant to minimize the injury done during an accident. And in still other procedures surgeons are able to improve a person's appearance by various plastic surgery techniques. Regardless of the purpose of the surgery, virtually all require some suturing of the skin and/or other tissues of the patient.

Depending on the type of surgery and the types of tissues involved, a surgeon may elect to use a variety of different suture needles. Common surgical needles include ⅝ circle, ½ circle, ⅜ circle, ¼ circle, J-shape, compound curve, ½ curved and straight. Each of these needles may also have a point type such as a taper point, blunt taper point, cutting edge, reverse cutting edge, taper cut and micro-point spatula. In the course of a surgery, a surgeon may elect any combination of needle shapes and points depending on the types of tissue which need to be closed and the types of incisions which were formed.

Additionally, there are numerous different suturing threads including polypropylene monofilament, polyester, polyester braided, nylon monofilament, silk, silk braded, and many others. These materials come in different sizes and are often color coded to allow the surgeon to verify what material he or she is working with. When combined with the different needle shapes and needle points, there are literally hundreds of potential combinations which may be used.

Because of the harm which has been caused from needles left in patients during surgeries, many states and virtually all hospitals require that every surgical suture be maintained in the operating field and accounted for before the patient is closed up at the end of the surgery. FIG. 1 shows a surgical tray 4 having a plurality of surgical suture packs 8a-8d disposed there on in accordance with principles of the prior art. Suture packs 8a-8d show the suture packs prior to opening. Inside the suture packs are usually a sterilized suture tray 12a-12j which is typically made from cardboard or plastic. Suture trays 12e-12j show suture trays for which the exterior packaging has been discarded.

The suture tray 12a-12j usually includes a needle 16e-16j and a length of suture thread 20e-20j of the desired material and thickness needed for the surgery. When a particular needle/suture thread is needed, the surgical technician in the operating room is responsible to grasp the desired needle with hemostats/forceps and hand it to the surgeon. When the surgeon is finished making the needed sutures, the needle is returned to the surgical technician who is responsible for securing the needle so that it can be used again as needed.

Surgical technicians are often also required to hold retractors and other instruments to assist the surgeon. Thus, the surgical technician may be required to secure the needle with one hand, while the other hand is being used to assist the surgeon with other tools. When a surgery is complex, the surgical technician may be dealing with ten or more different needle/suture combinations, as well as other surgical tools. Thus, it is easy to lose track of exactly which needle/suture combinations is in which location.

In an attempt to control the needles, many surgical technicians have resorted to using a sponge 30 into which the suture needles are secured when not in use. To further prevent the wrong needle/suture combination being given to the surgeon, some surgical technicians write on the sponge with a marker during surgery to indicate which needle/suture combination is which. It will be appreciated that to the naked eye, it is difficult to distinguish between a size 4 silk suture and a size 6 silk suture. While the size 6 silk suture may hold an incision closed, a size 3 or 4 may be more likely to break, thereby creating potential issues after the surgery. While writing the numbers helps identify which suture is which, it is also distracting during the surgery and if the surgical technician's attention is drawn away by the surgeon, it is easy to confuse the different needle/suture combinations.

While a surgical technician can write a code for the various needle/suture thread combinations which are likely to be used in advance, doing so requires the surgical technician to constantly determine which needles are disposed on the sponge, which packets have not yet been opened, and which needles are in use. This can be particularly confusing if a suture breaks when the needle is pulled from the suture tray 12. If, for example, a 4 silk ½ circle needle is pulled out and the suture breaks, the surgical technician may dispose the needle in the sponge at the number indicated. He or she must then get another 4 silk ½ circle package and open it for use. While the surgeon uses the needle, the location on the sponge for the 4 silk ½ circle is already filled—potentially leading to confusion. When there are unfilled numbers on the sponge, the technician must determine if the needle is missing or has yet to be used.

Additionally, at the end of the surgery, every suture needle must be accounted for. If one is missing, the surgical technician must figure out which one is missing and why. While knowing exactly which needle/suture combination is missing may help indicate places to look, this is only possible if one knows exactly which one is missing. Thus, there is a need for an improved method and apparatus for securing surgical needles.

### SUMMARY OF THE INVENTION

The following summary of the present invention is not intended to describe each illustrated embodiment or every possible implementation of the invention, but rather to give illustrative examples of application of principles of the invention.

A method and apparatus for holding surgical sutures may include disposing a suture needle holding pad on the suture tray or a cover thereof. The suture needle holding pad may be made from open cell or closed sell foam, Styrofoam, rubber, or other material which will receive and hold the needle in place. In a preferred embodiment, the surgical suture holding pad is made from a material which will not damage the tip of the needle when the needle is inserted therein and will allow the needle to be pulled out so that it can be used again.

### BRIEF DESCRIPTION OF THE DRAWINGS

Various embodiments of the present disclosure are shown and described in reference to the numbered drawings wherein:

FIG. 1 shows a surgical tray in accordance with the prior art;

FIG. 2 shows a top view of a surgical suture package in accordance with the principles of the present disclosure;

FIG. 3 shows a top view of the surgical suture tray of FIG. 2 with the packaging removed.

FIG. 4 shows a top view of the tray of FIG. 3 with the suture needle and suture thread removed;

FIG. 5 shows a side view of the suture tray of FIGS. 2-4;

FIG. 6 shows a top view of the surgical suture tray of FIGS. 2-5 with the suture needle attached to the surgical suture holding pad;

FIG. 7 shows a surgical tray having a number of surgical suture trays disposed thereon; and

FIG. 8 shows a method of securing a surgical suture needle on a suture tray.

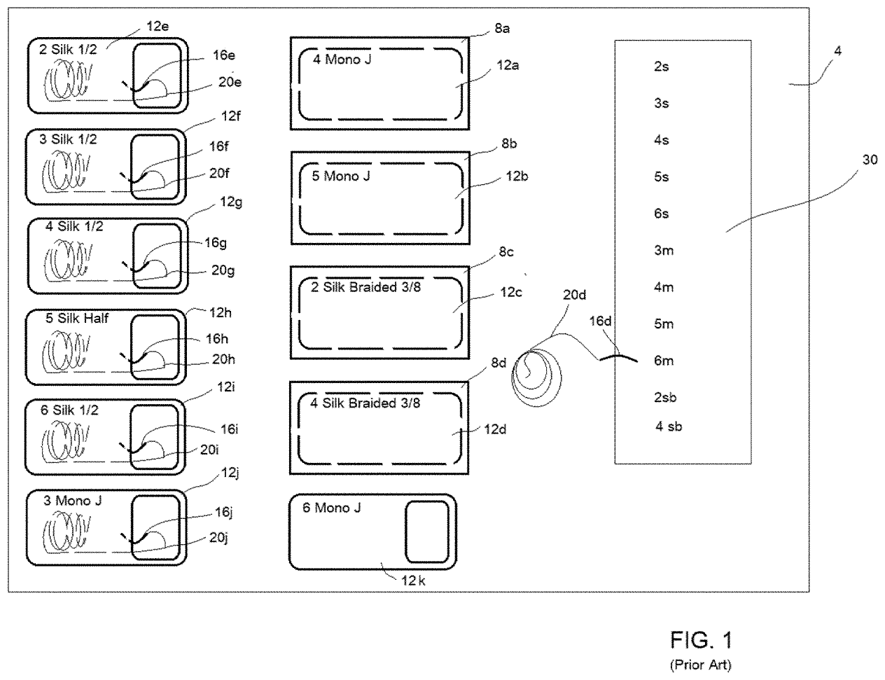

It will be appreciated that the drawings are illustrative and not limiting of the scope of the invention which is defined by the appended claims. The embodiments shown accomplish various aspects and objects of the invention. It will be appreciated that it is not possible to clearly show each element and aspect of the present disclosure in a single figure, and as such, multiple figures are presented to separately illustrate the various details of different aspects of the invention in greater clarity. Similarly, not all configurations or embodiments described herein or covered by the appended claims will include all of the aspects of the present disclosure as discussed above.

### DETAILED DESCRIPTION

Various aspects of the invention and accompanying drawings will now be discussed in reference to the numerals provided therein so as to enable one skilled in the art to practice the present invention. The skilled artisan will understand, however, that the methods described below can be practiced without employing these specific details, or that they can be used for purposes other than those described herein. Indeed, they can be modified and can be used in conjunction with products and techniques known to those of skill in the art in light of the present disclosure. The drawings and the descriptions thereof are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment,” “one configuration,” “an embodiment,” or “a configuration” means that a particular feature, structure, or characteristic described in connection with the embodiment may be included in at least one embodiment, etc. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the present disclosure may be combined in any suitable manner in one or more embodiments. In the following description, numerous specific details may be provided, such as examples of products or manufacturing techniques that may be used, to provide a thorough understanding of embodiments of the invention. One skilled in the relevant art will recognize, however, that embodiments discussed in the disclosure may be practiced without one or more of the specific details, or with other methods, components, materials, and so forth. In other instances, well-known structures, materials, or operations may not be shown or described in detail to avoid obscuring aspects of the invention.

Before the present invention is disclosed and described in detail, it should be understood that the present invention is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of that aspect in the claims.

It should also be noted that, as used in this specification and the appended claims, singular forms such as “a,” “an,” and “the” may include the plural unless the context clearly dictates otherwise. Thus, for example, reference to “a bracket” may include an embodiment having one or more of such brackets, and reference to “the target plate” may include reference to one or more of such target plates.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “generally” refers to something that has characteristics of a quality without necessarily being exactly that quality. For example, a structure said to be generally vertical would be at least as vertical as horizontal, i.e., would extend 45 degrees or greater from horizontal. Likewise, something said to be generally circular may be rounded like an oval but need not have a consistent diameter in every direction.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

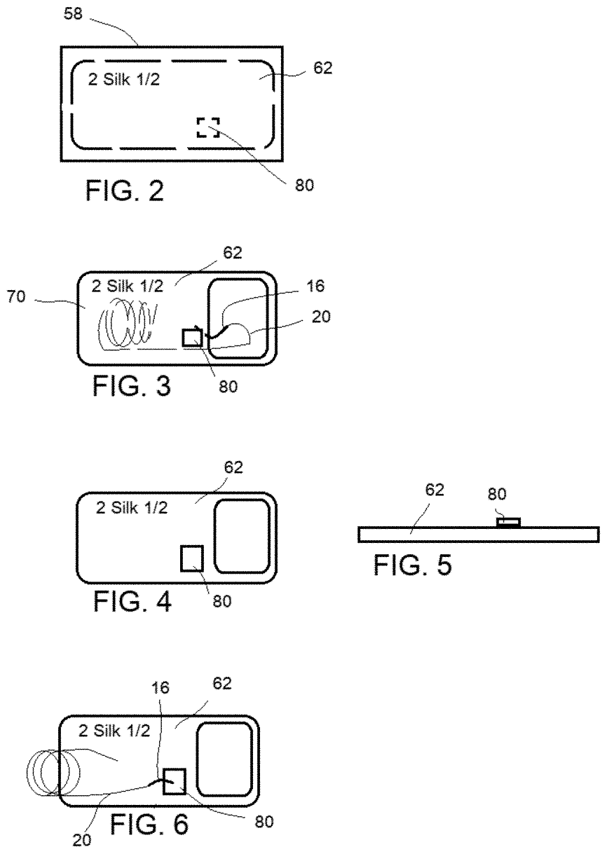

Turning now to FIG. 2, there is shown a surgical suture pack 58. The surgical suture pack 58 contains a suture tray 62. Disposed on the suture tray 62, or on a cover thereof, is a suture needle holding pad 80.

FIG. 3 shows the surgical suture tray 62 removed from the packaging. The surgical suture tray 62 holds a needle 16 and a suture thread 20. The suture tray may include an upper cover 70 which protects most of the suture material and which may have information associated with the needle and/or the suture thread disposed thereon. Thus, the suture thread is shown in dashed line. The needle 16 is often held in a small plastic holder below the upper cover so that the needle is partially covered by the upper cover 70 and is thus shown partially dashed. Other brand suture trays can be configured differently.

The suture needle holding pad 80 may be disposed on the surgical suture tray 62. (It will be appreciated in some current brands the suture tray is a small plastic tray and other brands use a folded piece of carboard to form the tray. The present disclosure is intended to include both these and a variety of other trays which may be used to carry a suture needle and suture thread.)

FIG. 4 shows the surgical suture tray 62 with the needle 16 and the suture thread 20 removed. Such would occur when the surgeon is using the suture needle 16 and suture thread 20 to suture a patient. When the suture is completed, the surgeon would hand the suture needle and suture thread back to the surgical technician. He or she would when secure the suture needle 16 into the suture needle holding pad 80 on the tray. Because each of the sutures would be attached to the suture tray 62 from which they are taken, the surgical technician can easily track the location of each suture needle and suture thread based on the information which is disposed on the suture tray. There is no need to use a separate sponge or for the surgical technician to try and write down which suture is which during the surgery. Moreover, in most situations, only one or at most two of the suture trays would be empty at any given time. Thus, the risk that a surgical technician might mistake one suture for another is significantly reduced. If a suture is missing, the surgical technician instantly knows which one is missing and a search based on when that suture was used can be undertaken.

FIG. 5 shows a side view of the surgical suture tray 62 and the suture needle holding pad 80. It will be appreciated that the suture needle holding pad 80 can be any shape and need only be thick enough to receive and hold the needle. It can be made from a variety of materials including foam, rubber, neoprene, fabric or any other sterilizable material which will receive and temporarily hold the needle without damaging the needle.

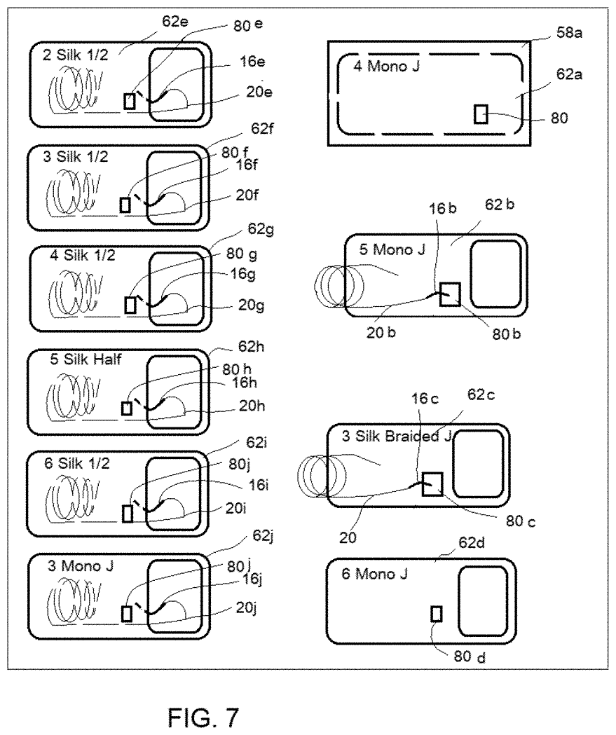

Turning now to FIG. 7, there is shown a surgical tray having a suture package 58a and suture trays 62b-62j made in accordance with the present disclosure. The surgical technician can readily determine which surgical trays have been opened and which suture packages are not yet opened. The surgical trays can even be arranged by which are open and which are not. By glancing at the suture trays 62b-62j, the surgical technician can tell that needles 16e through 16j have not yet been used. Needles 16b and 16c have been used and then secured in the suture needle holding pads 80b and 80c. Suture needle 16d (not shown) is currently in use and will be attached to the suture needle holding pad 80d when the surgeon returns it to the surgical technician. If a suture thread brakes, the needle can be attached to the associated suture needle holding pad and the surgical technician can see that the thread is broken. A second package of the same size and type may be opened and used without confusion at the sponge. Such an arrangement is far easier on the surgical technician and reduces work both before, during and at the end of the surgical procedure.

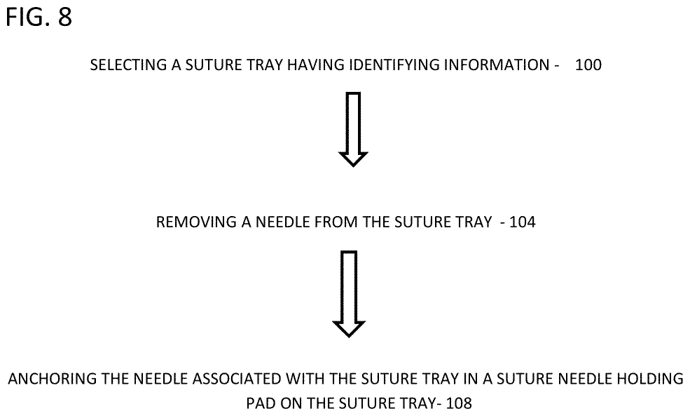

FIG. 8 shows a flow chart of steps which may be used in using and securing a surgical suture. The method involves selecting a suture tray 100. This may involve opening the packaging for a tray or selecting a tray which has been previously opened. The second step is removing the needle from the suture tray 104. This will typically involve removing the suture thread as well unless the suture thread breaks. The third step is anchoring the needle in the suture needle holding pad 108 when not in use by the physician. At appropriate times during the surgery the surgical technician can verify which needles have been removed but are not on their associated suture needle holding pads and can verify with the surgeon that he or she is using any needle that has been removed but it not on the pad.

Thus, there is disclosed a method and apparatus for holding suture needles. It will be appreciated that modifications can be made without departing from the scope and spirit of the invention. The appended claims are intended to cover such modifications.

1. An apparatus for holding a suture needle, the apparatus comprising:
a suture tray having a suture needle and suture material disposed in the tray, the suture needle being held to the tray by a needle holder; and
a label containing information about the needle and suture material disposed above the suture tray, the suture needle and the suture material being disposed below the label, the label having a suture needle holding pad disposed on the label of the suture tray.

2. The apparatus for holding a suture needle of claim 1, wherein the label forms the outermost upper surface of the apparatus apart from the suture needle holding pad while in use.

3. The apparatus for holding a suture needle of claim 1, wherein the suture tray, the label and the suture needle holding pad have been sterilized.

4. The apparatus for holding a suture needle of claim 3, wherein the suture tray is disposed in packaging, the packaging bearing information about at least one of a suture needle and suture thread contained therein.

5. (canceled)

6. The apparatus for holding a suture needle of claim 1, wherein
the suture needle holder holds the needle at least partially under the label.

7. The apparatus for holding a suture needle of claim 6 wherein the label forms a cover which has an opening and wherein the suture needle and at least some of the suture material are exposed in the opening.

8. The apparatus for holding a suture needle of claim 1, wherein the needle holder is made of plastic and the needle holding pad is made from foam.

9. The apparatus of claim 1, wherein the suture needle holding pad is disposed above the uppermost surface of the suture tray.

10. A method for securing suture needles, the method comprising:
selecting a suture tray having a suture needle and suture material disposed therein, the suture needle being mounted in a needle holder formed on the suture tray and a label attached to the suture tray, the label having a suture needle holding pad and information identifying the type of needle and suture material disposed thereon, the needle being at least partially disposed underneath the label;
removing the suture needle and suture material from the suture tray; and
inserting the suture needle into the suture needle holding pad attached to the label on the suture tray after using the suture needle so that the suture needle and suture material are held above the label by the suture needle holding pad.

11. The method according to claim 10, wherein the suture tray has information correlated to the suture needle and/or to the suture material disposed on the suture tray.

12. The method according to claim 10, wherein the method comprises selecting a suture tray having a suture needle holding pad disposed on a top side of the label thereof so that the suture needle holding pad is disposed above the suture tray.

13. The method according to claim 10, wherein the method comprises setting the suture tray on a surgical tray having a plurality of suture trays disposed thereon.

14. A surgical suture tray comprising:
a tray body having a surface having information written thereon identifying at least one of a suture needle and suture thread disposed therein;
a suture needle and suture thread disposed in the suture tray, the suture needle being held to the suture tray by a needle holder;
a label attached to the suture tray, the label having indicia disposed thereon identifying the suture needle and the suture thread, the label at least partially covering the needle; and
a suture needle holding pad disposed on top of the label, the suture needle holding pad being formed from a foam material for allowing the needle to be inserted into the needle holding pad to hold the suture needle to the top of the label after use.

15. (canceled)

16. (canceled)

17. (canceled)

18. The surgical suture tray according to claim 14, wherein the label includes information disposed thereon identifying a needle size, a needle shape, and a suture size.

19. The surgical suture tray according to claim 14, wherein the suture tray is disposed in a suture pack and wherein the suture tray is sterile prior to opening of the suture pack.

20. The surgical suture tray according to claim 19, wherein the suture needle holding pad is sterile prior to opening of the suture pack.

21. The apparatus for holding a suture needle of claim 1, wherein the label has an opening therein and wherein the needle is visible and retrievable through the opening, and wherein the suture needle holding pad disposed on the label is disposed near the opening.

22. The apparatus for holding the suture needle of claim 1, wherein the suture needle holding pad disposed on the label is square.

23. The surgical tray of claim 14, wherein information identifying the suture needle and suture thread are disposed on the tray body and wherein the needle holder is made of plastic.

24. The surgical tray of claim 14, wherein the suture needle is held to the suture tray below the label by a needle holder, the tip of the needle being exposed under the label.
